(19) United States
(12) Patent Application Publication
(10) Pub. No.: US 2025/0050326 A1
(43) Pub. Date: Feb. 13, 2025

(54) Hydrogels and Uses Thereof

(71) Applicant: The University of Liverpool, Liverpool (GB)

(72) Inventors: Srijan Jindal, Oxford (GB); Douglas Kell, Oxford (GB)

(21) Appl. No.: 18/722,887

(22) PCT Filed: Dec. 23, 2022
(86) PCT No.: PCT/GB2022/053395

§ 371 Date: Jun. 21, 2024

(30) Foreign Application Priority Data
Dec. 23, 2021 (GB) 2118945.1

(51) Int. Cl. B01L 3/00; C12Q 1/18

(52) U.S. Cl. CPC B01L 3/5023; C12Q 1/18; B01L 2200/16; B01L 2300/069; B01L 2300/0829; B01L 2300/0864; B01L 2400/0672; G01N 2800/348

(57) ABSTRACT

The present invention relates to A device for performing an assay on an aqueous biological sample, the device comprising: (i) at least a first chamber comprising a desiccated hydrogel, wherein the hydrogel incorporates at least a first desiccated assay reagent or reagents, the hydrogel and reagent or reagents being configured to allow the controlled release of the first reagent or reagents when the hydrogel has been exposed to the aqueous biological sample and has at least been partially hydrated; and (ii) an imaging device for imaging and analysing the first chamber after the aqueous biological sample has been introduced into the first chamber and the hydrogel has been at least partially hydrated and at least part of the first reagent or reagents has or have been released. The present invention also relates to a device for rapidly determining the susceptibility of a microorganism in an aqueous biological sample to an antimicrobial agent comprising: a) a multi-chamber plate, wherein: (i) at least a first chamber comprises a growth medium, a first dye, and a desiccated hydrogel; and (ii) at least a second chamber comprises a growth medium, a first dye, a desiccated hydrogel, and a first antimicrobial agent that inhibits or slows the proliferation of the, or a, microorganism; and b) an imaging device for imaging and analysing one or more chambers after the biological sample has been introduced into the chamber and the hydrogel has been at least partially hydrated. The invention also relates to related methods and a kit of parts. The device is particularly suited for identifying the which antimicrobial agents would be suitable for the treatment of microbial infections, such as Urinary Tract Infections (UTIs).

### TECHNICAL FIELD OF THE INVENTION

This invention relates to hydrogels and equipment and devices incorporating them for use in microfluidic diagnostic assays and rapid assays for detecting microbial presence and activity in biological samples. The invention is particularly suited for assessing antimicrobial susceptibility in biological samples from patients suspected of suffering from a microbial infection, and in particular for detecting antimicrobial susceptibility of bacteria in urine samples from patients suffering a Urinary Tract Infection (“UTI”).

### BACKGROUND TO THE INVENTION

Progress in the development of medical devices used for testing patient samples has accelerated in recent years due to advances in computing and the miniaturisation of electrical components. However, medical devices deployed in point of care settings still present an issue as the patient sample typically must be processed a certain way in order to prepare the sample before being presented to, and correctly analysed by, the device. The patient sample may be processed by adding the sample to reagents in a mixing vessel to form a mixture before presenting the mixture to the device for analysis. Frequently, the patient sample is not processed correctly by the healthcare professional. This either renders the sample unusable for analysis (requiring further samples to be taken from the patient) or can result in the wrong or incomplete diagnosis being given by the device. Furthermore, the act of mixing patient samples with reagents can pose a biohazard threat to the healthcare professional if adequate personal protective equipment is not (or incorrectly) deployed.

There are a number of medical conditions where a quick and reliable point of care medical device could make a dramatic difference to the correct diagnosis and result in the prescription of the correct medicine the first time. Microbial infections is one such area, where a physician often prescribes a course of a particular antimicrobial drug, despite not knowing whether the infection is of viral or bacterial origin and indeed whether it is a bacterial infection to which the antimicrobial drug is effective. The patient may return to physician after the course of antimicrobials if they have not worked, where the physician will then proceed to prescribe a different course of antimicrobials which may be effective. This approach not only wastes the time of both the patient and physician, but puts the patient at risk of the infection worsening and also contributes to the rise of resistance to antimicrobials.

What would be desirable would be a very rapid means of knowing, even before a patient left a doctor's surgery, that a particular antimicrobial was indeed capable of killing the organism causing the infection. While genotypic (whole-genome-sequencing) methods hold out some promise for this, what is really desired is a phenotypic assay that assesses the activity of anti-infectives in the sample itself.

Urinary tract infections (“UTIs”) are a worldwide patient problem. Other than in hospital-acquired infections, they are particularly common in females, with 1 in 2 women experiencing a UTI at some point in their life. Escherichia coli is the most common causative pathogen of a UTI. However, other Enterobacteriaceae such as Proteus mirabilis, Klebsiella spp. and Pseudomonas aeruginosa, and even Gram-positive cocci such as staphylococci and enterococci, may also be found (Kline and Lewis 2016; Tandogdu and Wagenlehner 2016).

E. coli cells in all conditions are highly heterogeneous (Kell et al. 2015), even if only because they are in different phases of the cell cycle (Wallden et al. 2016), and in both ‘exponential’ and stationary phase contain a variety of chromosome numbers (Åkerlund et al. 1995; Skarstad et al. 1986; Skarstad et al. 1985; Steen and Boye 1980; Stokke et al. 2012). To discriminate them physiologically, and especially to relate them to culturability (a property of an individual), it is necessary to study them individually (Kell et al. 1991; Taheri-Araghi et al. 2015), typically using flow cytometry. Flow cytometry has also been used to count microbes (and indeed white blood cells) for the purposes of assessing UTIs. Single cell morphological imaging has also been used, where in favourable cases antibiotic susceptibility can be detected in 15-30 minutes or less (Baltekin et al. 2017; Choi et al. 2014).

WO2020/109764 discloses method for rapidly determining the susceptibility of a microorganism to an antimicrobial agent using flow cytometry. Whilst the method was successful in quickly determining the susceptibility of a microorganism to an antimicrobial agent, flow cytometry is currently expensive and requires complex instrumentation and a significant amount of reagents to evaluate cell growth.

One object of the present invention is to provide a device for performing an assay on an aqueous sample which controls the flow of the biological sample and also allow the assay reagents to be released evenly.

It is another object of the present invention is to provide a device and media which can be used in rapid assays for detecting microbial presence and activity in biological samples. It is desirable that the device and media is simple to use and facilitate a fast and accurate assay which would enable a healthcare worker or physician to quickly assess microbial presence and activity in biological samples, the antimicrobial susceptibility of the microbe to a range of antimicrobials, and allow the prescription of an antimicrobial therapy that would successfully treat the microbial infection. Ideally, the device should be able to be used on a range of whole bodily fluids, such as blood, urine, mucus or saliva. It would also be desirable to provide a device for detecting antimicrobial susceptibility in urine from patients suffering from UTIs.

### SUMMARY OF THE INVENTION

In accordance with a first aspect of the present invention, there is provided, a device for performing an assay on an aqueous biological sample, the device comprising:
(i) at least a first chamber comprising a desiccated hydrogel, wherein the hydrogel incorporates at least a first desiccated assay reagent or reagents, and wherein the device comprises a second chamber comprising a desiccated hydrogel, wherein the hydrogel of the second chamber incorporates at least a second desiccated assay reagent or reagents and wherein the first and second chamber are connected to one or more conduits for enabling at least part of the aqueous biological sample to be fed into each chamber, the hydrogel and reagent or reagents being configured to allow the controlled release of the first reagent or reagents when the hydrogel has been exposed to the aqueous biological sample and has at least been partially hydrated; and
(ii) an imaging device for imaging and analysing the first chamber after the aqueous biological sample has been introduced into the first chamber and the hydrogel has been at least partially hydrated and at least part of the first reagent or reagents has or have been released.

The desiccated assay reagent will preferably be distributed evenly on, and/or throughout, the desiccated hydrogel.

Advantageously, the hydrogel controls the release of the reagent or regents. A common issue in devices employing microfluidics is that pre-coated reagents are dissolved too quickly when the microfluidic chamber is filled. That means the first volume of liquid entering the chamber dissolves more reagents than fluid entering later resulting in a concentration gradient. Distributing the desiccated assay reagent evenly on, and/or throughout, the desiccated hydrogel allows for a much improved controlled release of the reagent.

A further advantage of the hydrogel is when at least partially hydrated, it swells in volume, concentrating the solid and particulate matter in the chamber and also helping to expel bubbles from the chamber.

In certain embodiments, the device comprises a second chamber comprising a desiccated hydrogel, wherein the hydrogel incorporates the same desiccated assay reagent or reagents.

The first and second chamber may be connected to one another in a parallel arrangement. Alternatively, the first and second chamber are connected to one another in a series arrangement. It is preferred that the at least partial hydration of the hydrogel results in the hydrogel substantially preventing the flow of the aqueous biological sample between chambers. The prevention of flow of the aqueous biological sample between the chambers may be effected by the patrial hydration of the hydrogel blocking one or more conduit openings and essentially forming a plug.

Providing the hydrogel in the different chambers advantageously allows for the control of diffusion of regents between chambers in microfluidic applications, since the regents are only released by the hydrogel there is less chance of convective mixing occurring between neighbouring chambers.

The device may comprise an array of chambers.

The device may further comprise a heating arrangement adapted to apply heat to one or more chambers.

The imaging device may comprise a microscope.

The device may further comprises at least one transparent cover to cover a chamber and the imaging device may be adapted to be focused on the area of the chamber corresponding to the surface of hydrogel once partially or fully hydrated. Furthermore, the imaging device may be adapted to be focused on the area of the chamber between the transparent cover and the surface of hydrogel once partially or fully hydrated.

The imaging device may have a field of view of about 500 μm× about 700 μm or smaller.

The chamber may further comprise an optically contrasting filter paper on the desiccated hydrogel. Such a filter paper may be black. Alternatively, the chamber may further comprise low auto-fluorescence paper on the desiccated hydrogel.

The first desiccated assay reagents may comprise at least one bacterial growth medium, at least one fluorescent dye, and optionally, an antimicrobial agent.

In accordance with a second aspect of the present invention, there is provided a method for performing an assay on an aqueous biological sample, the method comprising the steps:
a) contacting:
   (i) a portion of the biological sample with a desiccated hydrogel in a first chamber, wherein the hydrogel incorporates at least a first desiccated assay reagent or reagents and the hydrogel and reagent or reagents being configured to allow the controlled release of the first reagent or reagents when the hydrogel has been exposed to the aqueous biological sample and has at least been partially hydrated; and
   (ii) another portion of the biological sample in a second chamber, wherein the hydrogel incorporates at least a second desiccated assay reagent or reagents and the hydrogel and reagent or reagents being configured to allow the controlled release of the second reagent or reagents when the hydrogel has been exposed to the aqueous biological sample and has at least been partially hydrated; and
   (iii) wherein the first and second chambers are connected to one or more conduits for enabling at least part of the aqueous biological sample to be fed into each chamber; and
b) incubating the samples in the first and second chambers for a period of time under conditions effective to enable the assay to be completed and to at least partially hydrate the hydrogel
c) imaging the first and second chambers and analysing the images to assess the results of the assay.

In accordance with a third aspect of the present invention, there is provided a kit of parts for forming a device for performing an assay on an aqueous biological sample, the kit comprising:
(i) at least a first chamber comprising a desiccated hydrogel, wherein the hydrogel incorporates at least a first desiccated assay reagent or reagents, the hydrogel and reagent or reagents being configured to allow the controlled release of the first reagent or reagents when the hydrogel has been exposed to the aqueous biological sample and has at least been partially hydrated; and
(ii) an imaging device for imaging and analysing the first chamber after the aqueous biological sample has been introduced into the first chamber and the hydrogel has been at least partially hydrated and at least part of the first reagent or reagents has or have been released.

The kit may be for use in producing a device herein above described with reference to the first aspect or for use in the method herein above described with reference to the third aspect.

In accordance with a fourth aspect of the present invention, there is provided, a device for rapidly determining the susceptibility of a microorganism in an aqueous biological sample to an antimicrobial agent comprising:
a) a multi-chamber plate, wherein:
   (i) at least a first chamber comprises a growth medium, a first dye, and a desiccated hydrogel; and
   (ii) at least a second chamber comprises a growth medium, a first dye, a desiccated hydrogel, and a first antimicrobial agent that inhibits or slows the proliferation of the, or a, microorganism; and
b) an imaging device for imaging and analysing one or more chambers after the biological sample has been introduced into the chamber and the hydrogel has been at least partially hydrated.

The provision of the combination of the growth medium, dye and desiccated hydrogel (and antimicrobial agent if present) in a single chamber, enables aqueous biological sample to be quickly tested as it contains all the reagents in the chamber for assessing growth, visualisation and antimicrobial susceptibility. Furthermore, the hydration of the hydrogel concentrates and presents the bacterial cells for imaging in a set focal plane for all chambers, which results in more accurate imaging and identification of bacterial cells and greatly reduced background signals. Indeed, the imaging device can utilise a relatively standard microscope if desired. The device of the present invention is relative simple and inexpensive to produce and enables the bacteria in a aqueous biological sample to be quickly identified and/or the susceptibility of the microorganism to multiple antimicrobial agents assessed. In a clinical setting, this would enable the physician to quickly identify which antimicrobial agent would be most successful in treating an infection.

In certain embodiments of the present invention, the desiccated hydrogel incorporates or is coated with a desiccated growth medium and/or a desiccated first dye.

The term, “aqueous biological sample” is intended to mean a sample derived from (or is) any bodily fluid (such as urine, blood, mucus or saliva for example), any environmental fluid (such as river or lake water or soil slurry), or any food or drink sample, which includes a component of water which is available for the hydration of the hydrogel.

The term, “hydrogel” is intended to mean any natural or synthetic hydrophilic polymer which does not dissolve in water.

In certain embodiments, the hydrogel comprises sodium polyacrylate gel or agar. Other hydrogels which are compatible with the invention may include sodium alginate, gelatine methacrylate, sodium polyacrylate, Matigel, synthetic peptide gels, agrose gel, polyacrylamide, fibrin gel and Laminin gels. It will of course be apparent to the skilled addressee that other hydrogels may also be employed. Preferably, the hydrogel hydrates to the desired volume within about 5 minutes, within about 4 minutes, within about 3 minutes, within about 2 minutes or within about 1 minute when in contact with the biological sample.

Most preferably, the hydrogel hydrates to the desired volume within about 2 minutes.

The device may further comprise a heating arrangement adapted to apply heat to one or more chambers or the whole of the plate.

The imaging device will preferably comprises a microscope. The microscope may additionally comprise or incorporate filters. Such filters could be either optical filters or software filters applied to the image data or a combination of both.

The device may further comprise one or more transparent covers to cover one or more chambers.

The imaging device may be adapted to be focused on the area of the chamber corresponding to the surface of hydrogel once partially or fully hydrated. Preferably, the imaging device is adapted to be focused on the area of the chamber between the transparent cover and the surface of hydrogel once partially or fully hydrated. The imaging device preferably has a field of view of about 750 μm× about 750 μm, about 500 μm× about 700 μm, about 400 μm×about 600 μm, about 300 μm× about 500 μm, about 200 μm× about 400 μm, or 100 μm×about 100 μm.

The dimensions of the chamber will vary depending upon application of the device. In some embodiments, the chamber may be in the range of about 10 mm×about 10 mm. In other embodiments, the chamber may be in the range of about 9 mm×about 9 mm, in the range of about 8 mm×about 8 mm, in the range of about 7 mm×about 7 mm, in the range of about 6 mm×about 6 mm or in the range of about 5 mm×5 mm. It will be obvious to the skilled address that the chamber may be any shape and not necessarily square in profile, Each chamber may further comprise an optically contrasting filter paper on the desiccated hydrogel. It is preferred that the filer paper is of a dark colour, such as black. Alternatively, each chamber may comprise low auto-fluorescence paper. In some embodiments, rather than providing optically contrasting filer paper or low auto-fluorescence paper, the hydrogel or the surface of a chamber may themselves be of a dark colour, such as black.

Advantageously, the present inventors found that using a black filter greatly reduced the background fluorescence and enhanced the signal to noise ratio by reducing the noise and enhances the fluorescent signal intensity for those bacterial cells which were ‘in focus’ and no fluorescent signal was produced for cells which were ‘out of focus’. The low background fluorescence with black filter paper therefore significantly assists in identifying the cells.

The dye will preferably comprise a fluorescent dye. In certain embodiments, a chamber may comprises two or more fluorescent dyes.

The plate may further comprise:
(i) at least a third chamber comprising a growth medium, a second dye, and a desiccated hydrogel; and
(ii) at least a fourth chamber comprising a growth medium, a second dye, a desiccated hydrogel, and a second antimicrobial agent that inhibits or slows the proliferation of the, or a, microorganism.

The imaging device may be operably coupled to an image analysis device. The imaging device may continuously or periodically analyse two or more chambers for bacterial cell number and/or bacterial morphology and/or fluorescence signal so as to determine the growth vs inhibition of growth or proliferation of a microorganism between two chambers containing the same growth media and dye and where only one of those chambers contains an antimicrobial agent.

The biological sample will preferably be derived from an individual believed to be suffering from a microorganism infection. The biological sample may be derived directly from potentially any body fluid, such as urine, blood, mucus or saliva. The biological samples may be whole or pre-treated with reagents or buffers or filtered prior to being contacted with chamber. In certain embodiments, the biological sample is urine.

The device as herein above described, may be for use in identifying the type or strain of microorganism infection in a biological sample. Preferably, the microorganism infection is a UTI.

In accordance with a fifth aspect of the present invention, there is provided a method for rapidly determining the susceptibility of a microorganism in an aqueous biological sample to an antimicrobial agent comprising the steps:
a) contacting:
   (i) a portion of the biological sample containing the microorganism with a growth medium, a first dye, and a desiccated hydrogel in a first chamber;
   (ii) another portion of the biological sample containing the microorganism with a growth medium, a first dye, a desiccated hydrogel, and a first antimicrobial agent that inhibits or slows the growth or proliferation of the, or a, microorganism in a second chamber by connecting the first and second chambers to one or more conduits for enabling at least part of the aqueous biological sample to be fed into each chamber;
b) incubating the samples in the first and second chambers for a period of time under conditions effective to enable or encourage growth or proliferation of the microorganism and to at least partially hydrate the hydrogel;
c) imaging the first and second chambers and analysing the images to assess the bacterial cell number and/or bacterial morphology and/or fluorescence signal of the bacterial cells in the first and second chambers during and/or after incubation so as to determine the growth or proliferation characteristics of the microorganism in the first chamber relative the inhibition of growth or inhibition of proliferation characteristics of the microorganism in the second chamber containing the antimicrobial agent; and
d) comparing the characteristics of the microorganisms in the first chamber with that of the second chamber during and/or after incubation, in order to establish the type or strain of microorganism and/or susceptibility of a microorganism to the antimicrobial agent.

The chambers may be heated during incubation. If the chambers are heated, it is preferred that the chambers are heated to a temperature in the range of about 35° C. and about 40° C. and preferably at a temperature of about 37° C.

Incubation step b) may be up to about 1 hour, up to about 55 minutes, up to about 50 minutes, up to about 45 minutes, up to about 40 minutes, up to about 35 minutes, up to about 30 minutes, up to about 25 minutes or up to about 20 minutes.

The chambers may be imaged using a microscope.

After the sample has been placed in contact with the chambers, the method may further comprise applying one or more transparent covers to cover the chambers.

The microscope may be adapted to be focused on the area of the chambers corresponding to the surface of the hydrogel once partially or fully hydrated.

The microscope may be adapted to be focused on the area of the chambers between the transparent cover and the surface of hydrogel once partially or fully hydrated. The microscope may have a field of view of about 750 μm× about 750 μm, about 500 μm× about 700 μm, about 400 μm× about 600 μm, about 300 μm× about 500 μm, about 200 μm× about 400 μm, or of about 100 μm× about 100 μm.

The dimensions of the chamber will vary depending upon application of the device. In some embodiments, the chamber may be in the range of about 10 mm×about 10 mm. In other embodiments, the chamber may be in the range of about 9 mm×about 9 mm, in the range of about 8 mm×about 8 mm, in the range of about 7 mm×about 7 mm, in the range of about 6 mm×about 6 mm or in the range of about 5 mm×5 mm. It will be obvious to the skilled address that the chamber may be any shape and not necessarily square in profile.

The method may further comprise applying optically contrasting filter paper over the hydrogel prior to contacting the samples with the chamber. Preferably, the filter paper is of a dark colour, such as black. Alternatively, the hydrogel or the surface of a chamber is of a dark colour, such as black.

The dye preferably comprises a fluorescent dye. In certain embodiments, the chamber comprises two or more fluorescent dyes.

Step a) of the method may further comprises contacting:
iii) another portion of the biological sample containing the microorganism with a growth medium, a second dye, a desiccated hydrogel in a third chamber;
iv) another portion of the biological sample containing the microorganism with a growth medium, a second dye, a desiccated hydrogel, and a second antimicrobial agent that inhibits or slows the growth or proliferation of the, or a, microorganism in a fourth chamber.

The chambers may be continuously or periodically imaged and analysed for bacterial cell number and/or bacterial morphology and/or fluorescence signal so as to determine the growth vs inhibition of growth or proliferation of a microorganism between two chambers containing the same growth media and dye and where only one of those chambers contains an antimicrobial agent. Alternatively, the chambers may simply be imaged and analysed after incubation step b).

The biological sample may be derived from an individual believed to be suffering from a microorganism infection. The biological sample may be urine and the microorganism infection may be a Urinary Tract Infection (UTI).

The method may be used for determining the antimicrobial agent for use in the treatment of a microorganism infection in an individual, wherein the method identifies which antimicrobial agent to administer to the individual based on comparing the bacterial cell number and/or bacterial morphology and/or fluorescence signal of the bacterial cells in two chambers containing the same growth media and dye and where only one of those chambers contains an antimicrobial agent.

In accordance with a sixth aspect of the present invention, there is provided a kit for rapidly determining the susceptibility of a microorganism in an aqueous biological sample to an antimicrobial agent comprising:
a) two or more chambers, wherein:
   (i) a first chamber comprises a growth medium, a first dye, a desiccated hydrogel;
   (ii) a second chamber comprises a growth medium, a first dye, a desiccated hydrogel, and a first antimicrobial agent that inhibits or slows the proliferation of the, or a, microorganism; and
   (iii) wherein the first and second chambers are connected to one or more conduits for enabling at least part of the aqueous biological sample to be fed into each chamber.

The kit may further comprise:
b) an imaging device for imaging and analysing one or more chambers after an aqueous biological sample has been introduced into the chamber and the hydrogel has been at least partially hydrated; and/or
c) a heating arrangement adapted to applying heat to one or more chambers.

The kit may be for use in producing a device herein above described with reference to the fourth aspect or for use in the method herein above described with reference to the fifth aspect.

Features, integers, characteristics, compounds, described in conjunction with a particular aspect, embodiment or example of the invention are to be understood to be applicable to any other aspect, embodiment or example described herein unless incompatible therewith. For example, the features, integers, characteristics of the fourth, fifth and sixth aspects will largely be applicable to, and interchangeable with the first, second and third aspects. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figures in which:

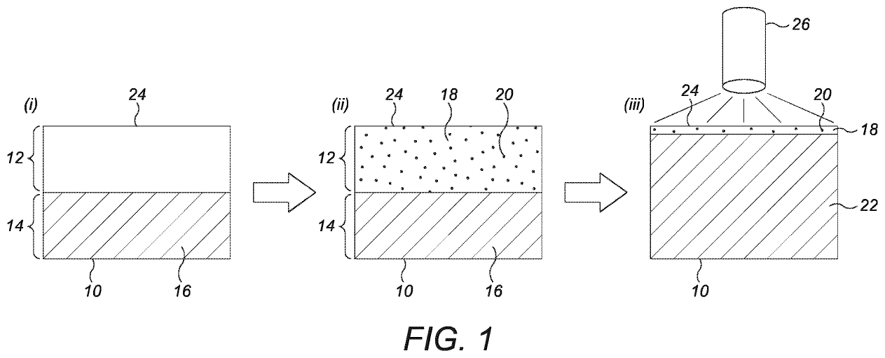

FIG. 1 is a schematic diagram of a chamber which incorporates a hydrogel in accordance with the present invention, where (i) shows the chamber before the biological sample is added, (ii) shows the chamber with the biological sample in situ and (iii) shows the chamber after the hydrogel has expanded.

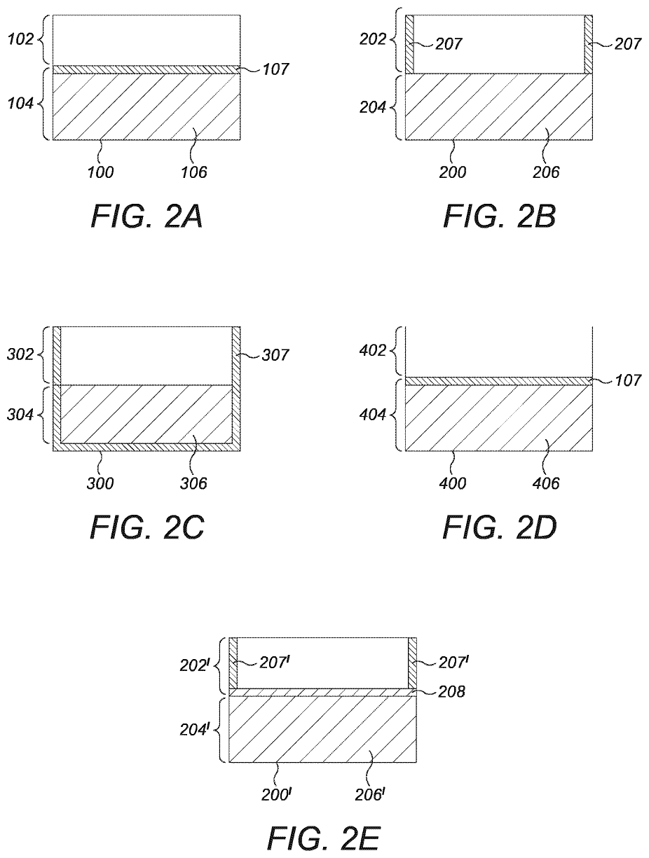

FIG. 2 shows alternative configurations of the chamber as shown in FIG. 1, where A shows a chamber with media coated on the top of the hydrogel, B shows a chamber with media coating the interior sides of the chamber, C shows a chamber with the media coating the interior side and bottom surfaces on the chamber, D shows a chamber where the media is on top of the hydrogel but where the chamber is open ended and E shows a chamber similar to that shown in B with the addition of black filter paper.

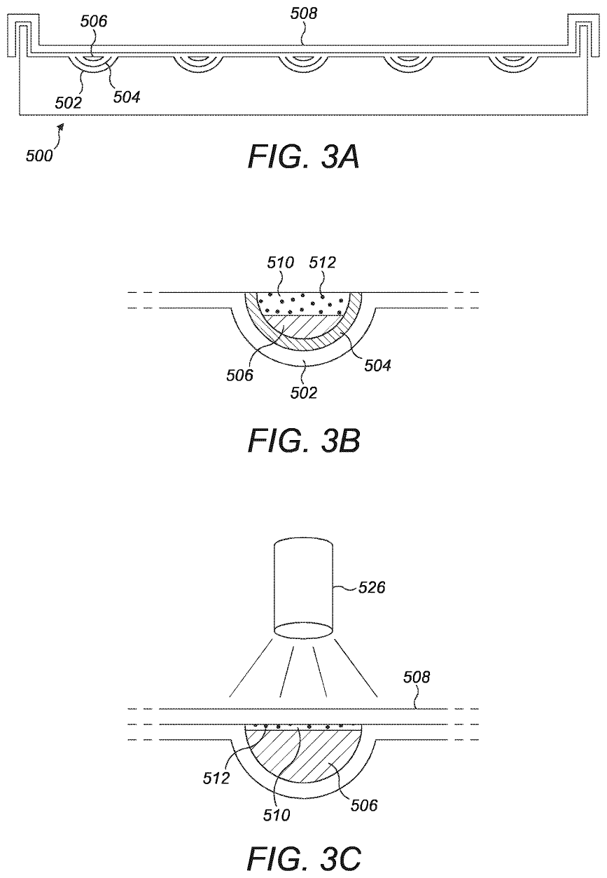

FIG. 3 shows a cross-sectional view of a multi well plate, and exploded views of an individual well, incorporating the hydrogel of the present invention, where A shows the view of a multi well plate, B shows a view of an individual well with the hydrogel and biological sample in situ, and C shows the individual well after the hydrogel has hydrated and is presented to an image capture device.

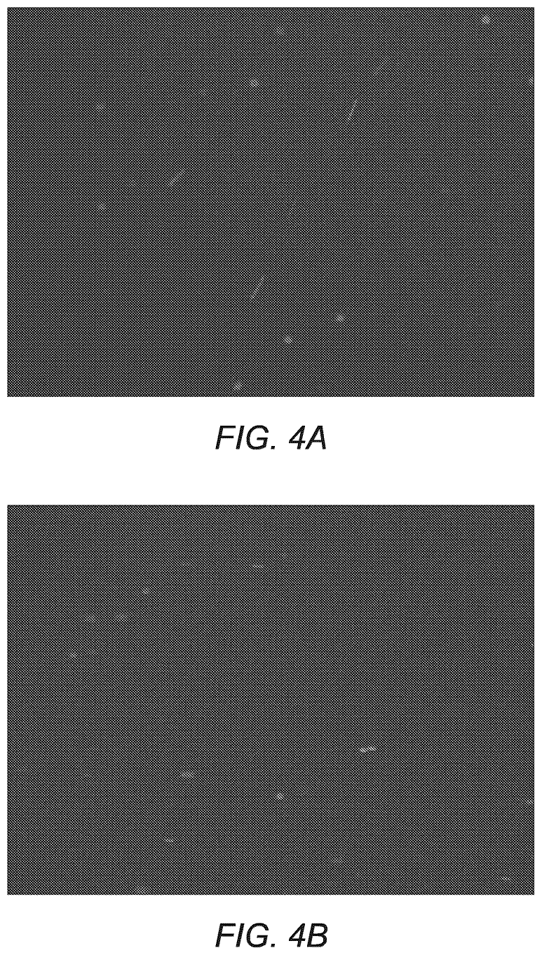

FIG. 4A is a first image of an aqueous GFP bead mixture using an optically filtered fluorescent microscope where the beads are in a liquid media.

FIG. 4B is a second image of an aqueous GFP bead mixture using an optically filtered fluorescent microscope where the beads are in a liquid media.

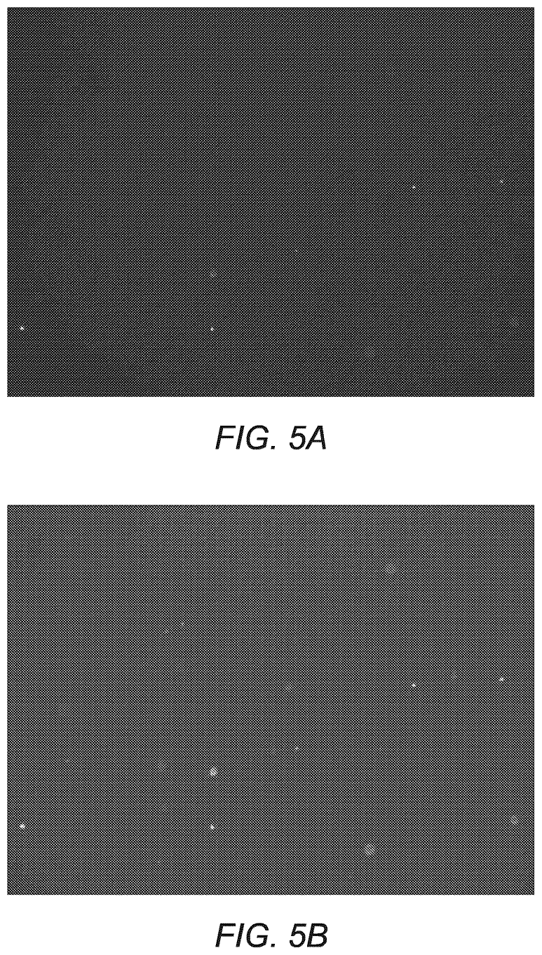

FIG. 5A is a first image of an aqueous GFP bead mixture using an optically filtered fluorescent microscope where the beads are embedded inside a hydrogel and a black filter was utilised.

FIG. 5B is a second image of an aqueous GFP bead mixture using an optically filtered fluorescent microscope where the beads are embedded inside a hydrogel.

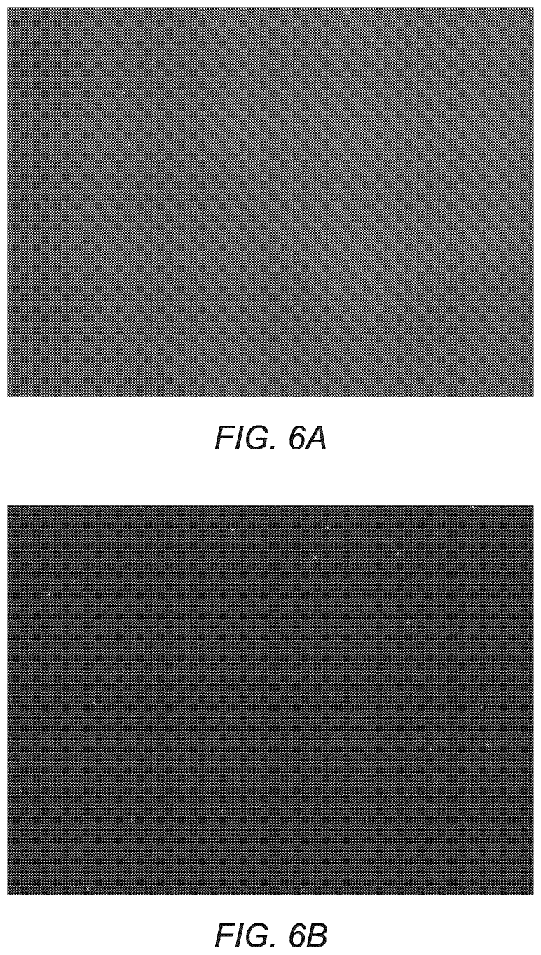

FIG. 6A is a first image of an aqueous GFP bead mixture using an optically filtered fluorescent microscope where the beads are on the surface of a hydrogel.

FIG. 6B is a second image of an aqueous GFP bead mixture using an optically filtered fluorescent microscope where the beads are on the surface of a hydrogel and a black filter was utilised.

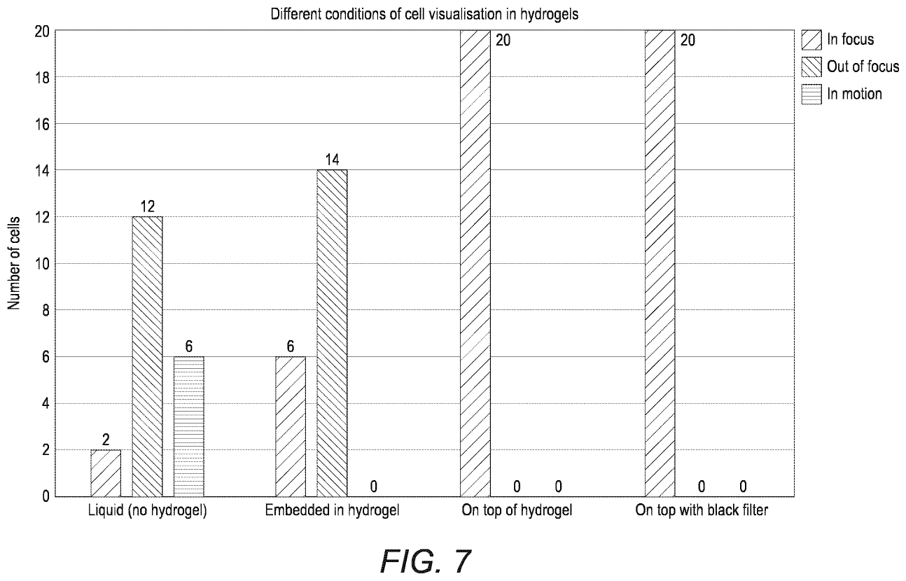

FIG. 7 is a graph showing the number of visual GFP beads identified as being ‘in focus’, ‘out of focus’ or ‘in motion’ by a fluorescent microscope in liquid culture, embedded in a hydrogel, on the surface of a hydrogel and on the surface of a hydrogel using a black filter.

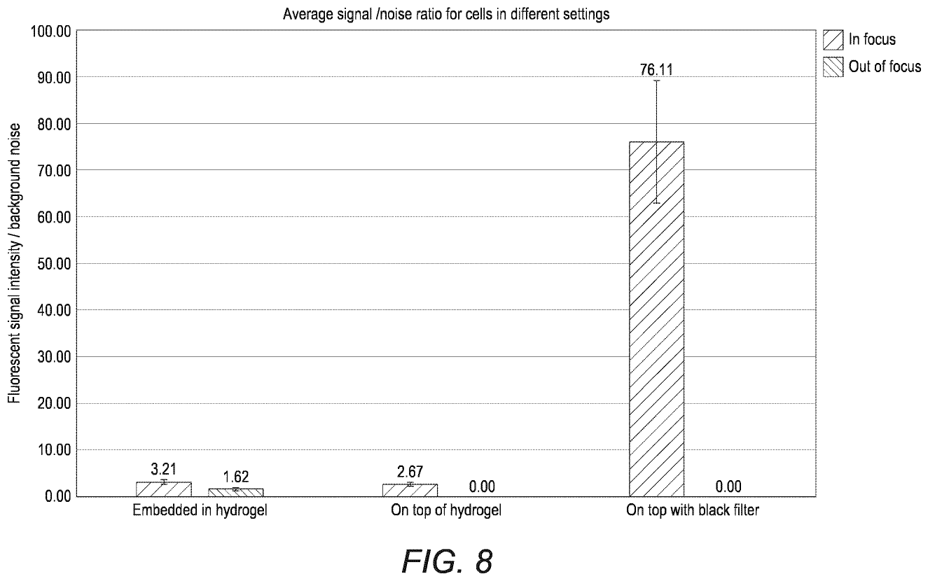

FIG. 8 is a graph showing the average fluorescent signal/noise ratio for GFP beads being ‘in focus’ or ‘out of focus’ by a fluorescent microscope when embedded in a hydrogel, on the surface of a hydrogel and on the surface of a hydrogel using a black filter.

### EXAMPLE 1

This example outlines embodiments which may be employed in accordance with the present invention.

With reference to FIG. 1, there is shown a chamber 10 having an upper portion 12 and lower portion 14. The lower portion 14 accommodates a desiccated hydrogel 16, whereas the upper portion 12 is for accommodating an aqueous biological sample, such as a urine sample 18. The urine sample 18 will be from a patient suffering from a urinary tract infection and therefore bacterial cells 20 are also present in the urine sample 18. At the top of the chamber 10 is a viewing portion 24, allowing an image captured device 24 to view the interior of the vessel 10 and capture images as required. The viewing portion is transparent and could be integrally formed with the chamber or a separate component such as cover slip or lid.

In use, a urine sample 18 from a patient suspected of suffering from a urinary tract infection is transferred to the upper portion 12 of the chamber 10. Immediately upon transferring the urine sample 18 to the chamber 10, the desiccated hydrogel 16 starts to absorb the liquid from the urine sample 18 and in doing so swells and expands, reducing the volume of liquid of the urine sample and concentrating the bacterial cells 20 into a much smaller volume of liquid at the top of the chamber 10. The image capture device 26 can then more accurately image the number and morphology of the bacterial cells 20 in order to help diagnose what type or strain of bacteria is causing the urinary tract infection.

The chamber 10 as shown in FIG. 1 may of course have a different cross-sectional shape than the chamber shown and may for example be in the shape of an arcuate well as typically found in multi well plates.

In order to allow the image capture device to accurately count and assess the morphology of the bacterial cells 20, the desiccated hydrogel 16 incorporates media, dyes and other reagents which may support (i.e. growth media) and/or inhibit (i.e. an antibiotic) the growth of a pre-determined bacterial strain in order to enable the assay to be performed.

With reference to FIG. 2A, there is shown a similar chamber to that shown in FIG. 1. The chamber 100 also incorporates a desiccated hydrogel 106 in the lower portion 104, but incorporates the media 107 on top of the desiccated hydrogel 106 at the interface between the upper portion 102 and the lower portion 104.

With reference to FIG. 2B, there is shown a chamber 200, having the desiccated hydrogel 206 in the lower portion 204, where the media 207 is coated to the sides of the upper portion 202.

With reference to FIG. 2C, there is shown a chamber 300 containing the desiccated hydrogel 306 in the lower portion 304, but the media 307 is coated on the sides of the upper portion 302, the lower portions 304 and also along the base of the chamber.

With reference to FIG. 2D there is shown a chamber 400 having a desiccated hydrogel 406 located in the lower portion 404 and the media 407 coating the top surface of the desiccated hydrogel 406 at the interface between the lower portion 404 and the upper portion 402. The chamber 10 is an open ended vessel and does not have a viewing portion as illustrated in FIG. 1, but still operates in a similar manner. However, rather than the bacterial cells being concentrated into a much smaller volume of liquid and being urged against the viewing portion, it simply enables bacteria to be concentrated and imaged more easily.

With reference to FIG. 2E, there is shown a chamber which is similar to that shown in FIG. 2B. Similarly, there is shown a chamber 200′, having the desiccated hydrogel 206′ in the lower portion 204′, that the media 207′ is coated to the sides of the upper portion 202. However, there is also provided a piece of thin black filter paper resting on top of the hydrogel 206′ which decreases background fluorescence and allows better image resolution by the image capture device. It will be apparent to the skilled addressee that in place of using black filter paper, the hydrogel and/or chamber walls could be coloured black in order to decrease background fluorescence and achieve a similar effect.

FIGS. 2A-2E, show different embodiments of chambers where the media 107, 207, 207′, 307, 407 can be applied to different parts of the chambers rather than incorporated in the hydrogel 106, 206, 206′, 306 and 406.

With reference to FIG. 3A, there is shown a cross-sectional view of a multi well plate 500, the multi well plate 500 may be a standard 96 (or other numbered well) or a bespoke multi well plate. The multi well plate 500 comprises a well 502 which is coated with media 504 and a desiccated hydrogel 506. The multi well plate 500 also has a lid 508 which can be applied over the induvial wells 502.

With reference to FIG. 3B, there is shown the cross-sectional view of the well 502, with the urine sample 510 being applied on top of the desiccated hydrogel 506. Upon applying the urine sample 510, the liquid in the sample dissolves and/or liquefies the media 504 which incorporates reagents to grow or in inhibit bacterial cells and imaging dyes and also hydrates the hydrogel 506.

With reference to FIG. 3C, the hydrogel 506 has been hydrated and the urine sample concentrated 510 so as to present the bacterial cells 512 close to (or adjacent to the lid 508 which may or may not comprise a viewing portion) so as to enable the image capture device 526 to be able to easily assess the quantity of bacterial cells 512 and/or any morphological or colour characteristics depending on the various dyes and growth or inhibiting agents which have been incorporated in the media 504. Each chamber may contain different dyes and/or reagents. For example, a first chamber may have a first dye and a first growth medium selected to stain and enable a known microorganism to proliferate and/or encourage the microorganism cell cycle to commence proliferation and a second chamber may contain a second dye and second growth medium which is substantially the same as the first dye and first growth medium but further comprises a antimicrobial agent is known to inhibit or slow the proliferation of the known microorganism. The chambers are repeatedly imaged using an automated microscope to count the number of viable bacteria present. The microscope will monitor several chambers that are pre-coated with culture media and fluorescent dyes, minimising reagent use. The use of dried reagents and a simple consumable makes this approach more practical for point of care applications By including multiple wells with different dyes and growth mediums with or without antimicrobial agents, a well plate can test a single urine sample for a range of types and strains of bacteria so as to establish which one is causing the urinary tract infection and which antibiotic to prescribe to the patient.

With reference to FIGS. 1-3, the various embodiments of the chambers are intended for illustrative purposes only and the skilled addressee will appreciate that all of them could be utilised in a device or system for the rapid detection of microbial presence and activity in biological samples more generally (such as blood samples) and they are not limited to solely assessing bacterial cells in urine samples.

### Example 2

Experiments were conducted to see whether the use of a hydrogel could result in improved identification of the number and morphology of bacterial cells. The experiments, investigated the image resolution using Green Fluorescent Protein (GFP) (488/530 nm) beads 1-3 μm in size (which are similar in size to the bacterial strains of interest): (i) in liquid media without using a hydrogel; (ii) embedded inside a hydrogel; and (iii) on top of a hydrogel. The hydrogels investigate were sodium polyacrylate and agar.

The following protocol was undertaken during these experiments.

Masks measuring 0.25 cm×0.25 cm were prepared with double-sided tape on a glass microscope slide so as to form chambers. One mask was not modified further, where sodium polyacrylate hydrogel was added to four masks and two covered with black filter paper.

10 μL of an aqueous mixture containing the GFP beads stained with 10× SYBR Green I was then applied to masks with coverslips, so as to form (i) a chamber containing GFP beads in liquid media without hydrogel; (ii) a chamber containing GFP beads in liquid media embedded inside the sodium polyacrylate hydrogel without black filter paper; (iii) a chamber containing GFP beadsin liquid media embedded inside the sodium polyacrylate hydrogel with black filter paper; (iv) a chamber containing GFP beads in liquid media on top of the sodium polyacrylate hydrogel without black filter paper; and (v) a chamber containing GFP beads in liquid media on top of the sodium polyacrylate hydrogel with black filter paper. For those cambers utilising a hydrogel, the gel was left for a period of approximately 2 minutes so as to absorb the bead mixture while the beads remain on the filter paper if present.

Each of the chambers were then visualised using a fluorescent microscope with the appropriate filters and images of the bacteria recorded.

FIGS. 4A and 4B shows images of the chamber containing beads in liquid media without sodium polyacrylate gel. The beads in this chamber could be identified, however the beads changed their position so monitoring them was difficult and the images were blurred and beads appeared as lines rather than static dots.

FIG. 5A shows images of the chamber containing beads in liquid media embedded inside a sodium polyacrylate gel with a black filter, whereas FIG. 5B shows images of the chamber containing beads in liquid media embedded inside a sodium polyacrylate gel without a black filter. The images show that the beads are static which allows them to be monitored more easily. Furthermore, the beads were in different focal planes and therefore harder to visualise and count. The size of imaged beads might also be different from their actual size because of their location in different plane.

FIG. 6A shows images of the chamber containing beads in liquid media on top of the sodium polyacrylate gel without a black filter. FIG. 6B shows images of the chamber containing beads in liquid media on top of the sodium polyacrylate gel with a black filter. The cells exhibited a slower growth rate. However, the cells were found to be static and easy to monitor. This approach also concentrated the beads allowing a smaller area to be scanned so as to capture the same number of beads. Furthermore, it was found that using a black filter paper (FIG. 6B) decreases the background fluorescence relative to not using a black filter paper (FIG. 6A).

FIG. 7 shows the bead count which were ‘in focus’, ‘out of focus’ or ‘in motion’ using the different chambers. It was found that for the beads in liquid media without a hydrogel, the beads were moving, whereas the all the beads were in focus when viewed on top of a hydrogel with or without a black filter.

FIG. 8 shows that the chamber containing beads in liquid media on top of the sodium polyacrylate gel with a black filter resulted in a greatly enhanced fluorescent signal intensity relative to the background noise for those beads which were ‘in focus’ and no fluorescent signal was produced for beads which were ‘out of focus’. The low background fluorescence with black filter paper therefore significantly assists in identifying the beads as compared to the background.

In practice, it is envisaged that an automated fluorescence microscope with a movable stage will be utilised and this will take sequences of images across all chambers which will be placed on a heater block to keep them at 37° C. Software for automated cell counting will also be employed in order to help automate the diagnosis for healthcare workers.

### Example 3

The experiments of Example 2 were repeated utilising a 1.5% agar hydrogel in place of the sodium polyacrylate gel and similar results were obtained and confirmed that utilising a chamber containing GFP beads in liquid media on top of a hydrogel with a black filter resulted in all beads being in focus and showing an enhanced fluorescent signal.

The forgoing embodiments are not intended to limit the scope of the protection afforded by the claims, but rather to describe examples of how the invention may be put into practice.

### REFERENCES

Åkerlund T, Nordström K, Bernander R: Analysis of cell size and DNA content in exponentially growing and stationary-phase batch cultures of Escherichia coli. J Bacteriol 1995; 177:6791-6797.
Baltekin O, Boucharin A, Tano E, Andersson D I, Elf J: Antibiotic susceptibility testing in less than 30 min using direct single-cell imaging. Proc Natl Acad Sci USA 2017; 114:9170-9175.
Choi J, Yoo J, Lee M, Kim E G, Lee J S, Lee S, Joo S, Song S H, Kim E C, Lee J C, Kim H C, Jung Y G, Kwon S: A rapid antimicrobial susceptibility test based on single-cell morphological analysis. Sci Transl Med 2014; 6: 267ra174.
Kell D B, Kaprelyants A S, Weichart D H, Harwood C L, Barer M R: Viability and activity in readily culturable bacteria: a review and discussion of the practical issues. Antonie van Leeuwenhoek 1998; 73:169-187.
Kell D B, Oliver S G: How drugs get into cells: tested and testable predictions to help discriminate between transporter-mediated uptake and lipoidal bilayer diffusion. Front Pharmacol 2014; 5:231.
Kline K A, Lewis A L: Gram-Positive Uropathogens, Polymicrobial Urinary Tract Infection, and the Emerging Microbiota of the Urinary Tract. Microbiology spectrum 2016; 4.
Skarstad K, Boye E, Steen H B: Timing of Initiation of Chromosome Replication in Individual Escherichia coli cells. EMBO Journal 1986; 5:1711-1717.
Skarstad K, Steen H B, Boye E: Escherichia coli DNA Distributions Measured by Flow Cytometry and Compared with Theoretical Computer Simulations. J Bacteriol 1985; 163:661-668.
Steen H B: Flow cytometric studies of microorganisms. In Melamed M R, Lindmo T, Mendelsohn ML (eds.): Flow Cytometry and Sorting (2nd Edition). New York: Wiley-Liss Inc., 1990:605-622.
Steen H B, Boye E: Bacterial growth studied by flow cytometry. Cytometry 1980; 1:32-36.
Stokke C, Flåtten I, Skarstad K: An easy-to-use simulation program demonstrates variations in bacterial cell cycle parameters depending on medium and temperature. PLOS One 2012; 7: e30981.
Taheri-Araghi S, Brown S D, Sauls J T, McIntosh D B, Jun S: Single-Cell Physiology. Annu Rev Biophys 2015; 44:123-142.
Tandogdu Z, Wagenlehner F M: Global epidemiology of urinary tract infections. Curr Opin Infect Dis 2016; 29:73-79.
Wallden M, Fange D, Lundius E G, Baltekin O, Elf J: The synchronization of replication and division cycles in individual E. coli cells. Cell 2016; 166:729-739.

1. A device for performing an assay on an aqueous biological sample, the device comprising:
(i) at least a first chamber comprising a desiccated hydrogel, wherein the hydrogel incorporates at least a first desiccated assay reagent or reagents, and wherein the device comprises a second chamber comprising a desiccated hydrogel, wherein the hydrogel of the second chamber incorporates at least a second desiccated assay reagent or reagents and wherein the first and second chamber are connected to one or more conduits for enabling at least part of the aqueous biological sample to be fed into each chamber, the hydrogel and reagent or reagents being configured to allow the controlled release of the first and second reagent or reagents when the hydrogel has been exposed to the aqueous biological sample and has at least been partially hydrated; and
(ii) an imaging device for imaging and analysing the first chamber after the aqueous biological sample has been introduced into the first chamber and the hydrogel has been at least partially hydrated and at least part of the first reagent or reagents has or have been released.

2. The device according to claim 1, wherein the desiccated assay reagent is distributed evenly on, and/or throughout, the desiccated hydrogel.

3. The device according to claim 1, wherein the device comprises a second chamber comprising a desiccated hydrogel, wherein the hydrogel incorporates the same desiccated assay reagent or reagents and/or the first and second chamber are connected to one another in a parallel arrangement and/or series arrangement.

4-5. (canceled)

6. The device according to claim 35, wherein the at least partial hydration of the hydrogel results in the hydrogel substantially preventing the flow of the aqueous biological sample between chambers and/or the prevention of flow of the aqueous biological sample between the chambers is effected by the patrial hydration of the hydrogel blocking one or more conduit openings.

7. (canceled)

8. The device according to claim 1, wherein the device comprises an array of chambers and/or a heating arrangement adapted to apply heat to one or more chambers.

9. (canceled)

10. The device according to claim 1, wherein the imaging device comprises a microscope and/or the device further comprises at least one transparent cover to cover a chamber.

11-13. (canceled)

14. The device as claimed in claim 1, wherein the imaging device has a field of view of about 500 μm× about 700 μm.

15. The device according to claim 1, wherein the chamber further comprises an optically contrasting filter paper on the desiccated hydrogel.

16. (canceled)

17. The device according to claim 1, wherein the chamber further comprises low auto-fluorescence paper on the desiccated hydrogel.

18. The device according to claim 1, wherein the first desiccated assay reagents comprise at least one bacterial growth medium, at least one fluorescent dye, and optionally, an antimicrobial agent.

19. A device for rapidly determining the susceptibility of a microorganism in an aqueous biological sample to an antimicrobial agent comprising:
a) a multi-chamber plate, wherein:
(i) at least a first chamber comprises a growth medium, a first dye, and a desiccated hydrogel; and
(ii) at least a second chamber comprises a growth medium, a first dye, a desiccated hydrogel, and a first antimicrobial agent that inhibits or slows the proliferation of the, or a, microorganism; and
(iii) wherein the first and second chambers are connected to one or more conduits for enabling at least part of the aqueous biological sample to be fed into each chamber; and
b) an imaging device for imaging and analysing one or more chambers after the biological sample has been introduced into the chamber and the hydrogel has been at least partially hydrated.

20. The device according to claim 19, wherein the device further comprises a heating arrangement adapted to apply heat to one or more chambers or the whole of the plate.

21. The device according to claim 19, wherein the imaging device comprises a microscope and/or the device further comprises one or more transparent covers to cover one or more chambers.

22-24. (canceled)

25. The device as claimed in claim 19, wherein the imaging device has a field of view of about 500 μm× about 700 μm.

26. The device according to claim 19, wherein each chamber further comprises an optically contrasting filter paper on the desiccated hydrogel.

27. (canceled)

28. The device according to claim 19, wherein each chamber further comprises low auto-fluorescence paper on the desiccated hydrogel.

29. The device according to claim 19, wherein the hydrogel or the surface of a chamber is black.

30. The device according to claim 19, wherein the dye comprises a fluorescent dye and/or a chamber comprises two or more fluorescent dyes.

31. (canceled)

32. The device according to claim 19 preceding claim, wherein the plate further comprises:
(i) at least a third chamber comprising a growth medium, a second dye, and a desiccated hydrogel; and
(ii) at least a fourth chamber comprising a growth medium, a second dye, a desiccated hydrogel, and a second antimicrobial agent that inhibits or slows the proliferation of the, or a, microorganism.

33. The device according to claim 19, wherein the imaging device is operably coupled to an image analysis device.

34. The device according to claim 19, wherein the imaging device continuously or periodically analyse two or more chambers for bacterial cell number and/or bacterial morphology and/or fluorescence signal so as to determine the growth vs inhibition of growth or proliferation of a microorganism between two chambers containing the same growth media and dye and where only one of those chambers contains an antimicrobial agent.

35. The device according to claim 19, wherein the biological sample is derived from an individual believed to be suffering from a microorganism infection and/or the biological sample is urine.

36. (canceled)

37. The device as claimed in claim 19, for use in identifying the type or strain of microorganism infection in a biological sample.

38. (canceled)

39. A method for rapidly determining the susceptibility of a microorganism in an aqueous biological sample to an antimicrobial agent comprising the steps:
a) contacting:
(i) a portion of the biological sample containing the microorganism with a growth medium, a first dye, and a desiccated hydrogel in a first chamber; and
(ii) another portion of the biological sample containing the microorganism with a growth medium, a first dye, a desiccated hydrogel, and a first antimicrobial agent that inhibits or slows the growth or proliferation of the, or a, microorganism in a second chamber by connecting the first and second chambers to one or more conduits for enabling at least part of the aqueous biological sample to be fed into each chamber;
b) incubating the samples in the first and second chambers for a period of time under conditions effective to enable or encourage growth or proliferation of the microorganism and to at least partially hydrate the hydrogel;
c) imaging the first and second chambers and analysing the images to assess the bacterial cell number and/or bacterial morphology and/or fluorescence signal of the bacterial cells in the first and second chambers during and/or after incubation so as to determine the growth or proliferation characteristics of the microorganism in the first chamber relative the inhibition of growth or inhibition of proliferation characteristics of the microorganism in the second chamber containing the antimicrobial agent; and
d) comparing the characteristics of the microorganisms in the first chamber with that of the second chamber during and/or after incubation, in order to establish the type or strain of microorganism and/or susceptibility of a microorganism to the antimicrobial agent.

40. The method according to claim 39, wherein the chambers are heated during incubation and/or the chambers are heated to a temperature in the range of about 35° C. and about 40° C.

41-61. (canceled)
